(19) United States
(12) Patent Application Publication
(10) Pub. No.: US 2024/0350431 A1
(43) Pub. Date: Oct. 24, 2024

(54) COMPOSITIONS AND METHODS FOR TREATMENT OF ALOPECIA AREATA

(71) Applicant: VERADERMICS INCORPORATED, West Hartford, CT (US)

(72) Inventors: Reid WALDMAN, West Hartford, CT (US); Keith Arthur JOHNSON, Durham, NC (US)

(21) Appl. No.: 18/686,168

(22) PCT Filed: Aug. 22, 2022
(86) PCT No.: PCT/US2022/041048

§ 371 Date: Feb. 23, 2024

Related U.S. Application Data
(60) Provisional application No. 63/286,987, filed on Dec. 7, 2021.
(60) Provisional application No. 63/284,231, filed on Nov. 30, 2021.
(60) Provisional application No. 63/235,999, filed on Aug. 23, 2021.

(51) Int. Cl. A61K 31/122; A61M 35/00

(52) U.S. Cl. CPC A61K 31/122; A61M 35/003

(57) ABSTRACT

The application provides a shelf stable squaric acid ester composition (SAE). The application further provides a system and method for delivering a SAE composition to a subject to treat alopecia areata. The system comprises a storage compartment containing SAE and an excipient and an applicator unit for topical administration of the SAE composition.

This application is a U.S. National Stage filing under 35 U.S.C. § 371 of International PCT Application No. PCT/US2022/041048, filed Aug. 22, 2022 which claims priority to U.S. Provisional Application No. 63/235,999 filed on Aug. 23, 2021, U.S. Provisional Application No. 63/284,231 filed on Nov. 30, 2021, and U.S. Provisional Application No. 63/286,987 filed on Dec. 7, 2021, which are incorporated herein by reference in their entirety.

### SUMMARY

Embodiments of the application are directed to a system for delivering a squaric acid ester (SAE) composition to a subject comprising: a storage compartment containing the composition having at least about 2.5% (w/v) SAE and an excipient; and an applicator unit, wherein the applicator unit delivers a volume of about 1 milliliter of the SAE composition.

Embodiments of the present application are directed to a method of delivering a squaric acid ester (SAE) composition to a subject for treating a condition susceptible to treatment with the SAE composition, comprising: providing applicator device comprising a storage compartment, an air tight seal, a connecting region, and an applicator; and delivering the SAE composition from the storage compartment through the applicator to the subject.

Further embodiments of the present application are directed to a kit for administering a squaric acid ester (SAE) composition to a subject, comprising: an applicator device comprising a storage compartment, an air tight seal, a connecting region, and an applicator; said kit further comprising instructional material for administering said SAE composition for treating a condition susceptible to treatment using said SAE composition.

Further embodiments of the present application are directed to a composition comprising: squaric acid ester (SAE); and an excipient; wherein said composition is essentially free of water and DMSO; and wherein said composition has a total volume of 1 milliliter.

### BRIEF DESCRIPTION OF THE DRAWINGS

FIG. 1 is a schematic representation of an embodiment of a system for delivering a squaric acid ester (SAE) composition to a subject.

### DETAILED DESCRIPTION

This disclosure is not limited to the particular systems, devices and methods described, as these may vary. The terminology used in the description is for the purpose of describing the particular versions or embodiments only, and is not intended to limit the scope. Such aspects of the disclosure be embodied in many different forms; rather, these embodiments are provided so that this disclosure will be thorough and complete, and will fully convey its scope to those skilled in the art.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

All percentages, parts and ratios are based upon the total weight of the compositions and all measurements made are at about 25° C., unless otherwise specified.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation may be modified as would be readily apparent to a person skilled in the art. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The term “substantially free” or “essentially free” as used herein indicates that a specified substance referred to is present in amounts not more than 10% by weight or volume of the total composition. The term “essentially free of water” as used herein indicates that the composition is essentially free from any source of water, including atmospheric water.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “animal” as used herein includes, but is not limited to, humans and non-human vertebrates such as wild, domestic, and farm animals.

The term “disorder” is used in this disclosure to mean, and is used interchangeably with, the terms disease, condition, or illness, unless otherwise indicated.

The terms “administer,” “administering” or “administration” as used herein refer to either directly administering a compound (also referred to as an agent of interest) or pharmaceutically acceptable salt of the compound (agent of interest) or a composition to a subject.

The term “treat,” “treated,” or “treating” as used herein refers to both therapeutic treatment and prophylactic or preventative measures, wherein the object is to reduce the frequency of, or delay the onset of, symptoms of a medical condition, enhance the texture, appearance, color, sensation, or hydration of the intended tissue treatment area of the tissue surface in a subject relative to a subject not receiving the compound or composition, or to otherwise obtain beneficial or desired clinical results. For the purposes of this invention, beneficial or desired clinical results include, but are not limited to, reversal, reduction, or alleviation of symptoms of a condition; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

In some embodiments, the compounds and methods disclosed herein can be utilized with or on a subject in need of such treatment, which can also be referred to as “in need thereof.” As used herein, the phrase “in need thereof” means that the subject has been identified as having a need for the particular method or treatment and that the treatment has been given to the subject for that particular purpose.

As used herein, the term “therapeutic” or “therapeutic agent” or “pharmaceutically active agent” means an agent utilized to treat, combat, ameliorate, prevent or improve an unwanted condition or disease of a patient.

The term “composition” as used herein refers to a combination or a mixture of two or more different ingredients, components, or substances.

The term “controlled delivery” as used herein refers to the delivery of the SAE composition to a particular location or for a specific amount of time to the skin of a patient. The term “precise” as used herein refers to the delivery of the SAE composition to an accurate location or an accurate amount of composition. The term “precise” refers to a location or time that deviates not more than 10% from the desired location or volume.

The phrase “therapeutically effective” is intended to qualify the amount of active ingredients (i.e, the compounds or derivatives thereof) used in the treatment of a disease or disorder or on the effecting of a clinical endpoint.

A “therapeutically effective amount” or “effective amount” of a compound or composition is a predetermined amount calculated to achieve the desired effect, i.e., to inhibit, block, or reverse the activation, migration, or proliferation of cells. The activity contemplated by the present methods includes both medical therapeutic and/or prophylactic treatment, as appropriate. The specific dose of a compound administered according to this invention to obtain therapeutic and/or prophylactic effects will, of course, be determined by the particular circumstances surrounding the case, including, for example, the compound administered, the route of administration, and the condition being treated. The compounds are effective over a wide dosage range. However, it will be understood that the effective amount administered will be determined by the physician in the light of the relevant circumstances including the condition to be treated, the choice of compound to be administered, and the chosen route of administration, and therefore the above dosage ranges are not intended to limit the scope of the invention in any way. A therapeutically effective amount of compound of this invention is typically an amount such that when it is administered in a physiologically tolerable excipient composition, it is sufficient to achieve an effective systemic concentration or local concentration in the tissue.

The term “responder” as used herein refers to a subject or set of subjects that demonstrate an improvement in symptoms after treatment with a therapeutically effective amount of a SAE composition. The term “partial responder” as used herein refers to a subject or set of subjects that experience measurable hair regrowth at any amount less than about a 50% SALT score at the conclusion of four months of treatment.

The term “non-responder” as used herein refers to a subject or set of subjects that do not demonstrate an improvement in symptoms after treatment with a therapeutically effective amount of a SAE composition.

The term “inhibiting” includes the administration of a compound of the present invention to prevent the onset of the symptoms, alleviating the symptoms, reducing the symptoms, delaying or decreasing the progression of the disease and/or its symptoms, or eliminating the disease, condition or disorder.

As used herein, the phrase “essentially no water” refers to an amount of water that would not lead to hydrolysis of a SAE composition into its free acid form which is clinically inactive. “Essentially no atmospherics water” may be defined as about 10% or less of water.

The term “excipients” as used herein encompasses carriers and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The terms “topically” and “topical” as used herein refer to application of the compositions to the surface of the skin, mucosal cells, keratins and tissues. Examples of keratins are nails and hair.

The term “skin” as used herein refers to the thin layer of tissue forming the natural outer covering of the body of a person or animal. The skin is made of the epidermis and dermis. As used herein, the stratum corneum is the outermost layer of the epidermis and the papillary dermis is the uppermost layer of the dermis. The term “scalp” as used herein refers to the skin on a patient's head.

The term “stable” as used herein refers to a formulation which retains the same properties and characteristics that is possessed at the time of manufacturing after storage of the formulation. Properties may be chemical, physical, microbiological, therapeutic, and toxicological. A stable formulation retains 90% of the original property of interest after storage.

The term “shelf stable” as used herein refers to a formulation which is stable under standard environmental and atmospheric conditions for up to about 60 months. Standard environmental and atmospheric conditions may include temperatures ranging from 4° C. to about 50° C.

The term “room temperature” as used herein refers to “controlled room temperature” as between 20 degrees Celsius to 25 degrees C. Celsius, which is equivalent to 68 degrees Fahrenheit to 77 degrees Fahrenheit, with excursions between 15 degrees Celsius to 30 degrees Celsius, which is equivalent to 59 degrees Fahrenheit to 86 degrees Fahrenheit, allowed for a mean temperature range of about 68 degrees Fahrenheit to 77 degrees Fahrenheit.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason. Throughout this disclosure, various patents, patent applications and publications are referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

### A System for Delivering a SAE Composition

Embodiments of the application are directed to a system for delivering a squaric acid ester (SAE) composition to a subject comprising: a storage compartment containing the composition having at least about 2.5% (w/v) SAE and an excipient; and an applicator unit, wherein the applicator unit delivers a volume of about 1 milliliter of the SAE composition.

In some embodiments, the SAE composition comprises squaric acid dibutylester (SADBE). In some embodiments, the SAE composition comprises squaric acid diethylester (SADEE). In some embodiments, the SAE composition comprises a single SAE. In some embodiments, the SAE composition comprises a combination of SAEs. In some embodiments, the SEA composition comprises SADBE and SADEE.

In some embodiments, the system for delivering a SAE composition to a subject comprises at least about 2.5% (w/v) SAE. In some embodiments, the system for delivering a SAE composition to a subject comprises at least about 5% (w/v) SAE. In some embodiments, the system for delivering a SAE composition to a subject comprises at least about 7% (w/v) SAE. In some embodiments, the system for delivering a SAE composition to a subject comprises at least about 10% (w/v) SAE. In some embodiments, the system for delivering a SAE composition to a subject comprises a range of SAE from about 1% to about 20%, about 1% to about 18%, about 1% to about 16%, about 1% to about 15%, about 1% to about 14%, about 10% to about 12%, about 10% to about 10%, about 10% to about 9%, about 1% to about 8%, about 1% to about 7%, about 1% to about 6%, about 1% to about 5%, about 1% to about 4%, about 1% to about 3%, about 1% to about 2%, about 2% to about 20%, about 3% to about 20%, about 4% to about 20%, about 5% to about 20%, about 6% to about 20%, about 7% to about 20%, about 8% to about 20%, about 9% to about 20%, about 10% to about 20%, about 12% to about 20%, about 14% to about 20%, about 15% to about 20%, about 16% to about 20%, about 18% to about 20%, or a value within these ranges. Specific examples may include about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20% or a range between any two of these values.

Some embodiments disclosed herein also include pharmaceutical compositions which contain, as the active ingredient, one or more of the compounds disclosed herein in combination with one or more pharmaceutically acceptable carriers (excipients). In some embodiments, the excipient may be selected from at least one of the following: ketones, alcohols, other organic solvents.

In some embodiments, the system for delivering a SAE composition to a subject comprises delivering a therapeutically effective volume of the SAE composition. In some embodiments, the system for delivering a SAE composition to a subject comprises delivering an effective volume of the SAE composition. In some embodiments, the system for delivering a SAE composition to a subject comprises delivering about 1 milliliter of the SAE composition. In some embodiments, the system for delivering a SAE composition to a subject comprises delivering about 0.8 milliliters of the SAE composition. In some embodiments, the system for delivering a SAE composition to a subject comprises delivering about 1.2 milliliters of the SAE composition. In some embodiments, the system for delivering a SAE composition to a subject comprises delivering a range of about 0.7 milliliters to about 1.3 milliliters of the SAE composition. In some embodiments, the system for delivering a SAE composition to a subject comprises delivering a range of about 0.1 milliliters to about 2 milliliters, about 0.1 milliliters to about 1.9 milliliters, about 0.1 milliliters to about 1.8 milliliters, about 0.1 milliliters to about 1.7 milliliters, about 0.1 milliliters to about 1.6 milliliters, about 0.1 to about 1.5 milliliters, about 0.1 milliliters to about 1.4 milliliters, about 0.1 milliliters to about 1.3 milliliters, about 0.1 milliliters to about 1.2 milliliters, about 0.1 milliliters to about 1.1 milliliters, about 0.1 milliliters to about 1.0 milliliters, about 0.1 milliliters to about 0.9 milliliters, about 0.1 milliliters to about 0.8 milliliters, about 0.1 milliliters to about 0.7 milliliters, about 0.1 milliliters to about 0.6 milliliters, about 0.1 milliliters to about 0.5 milliliters, about 0.1 milliliters to about 0.4 milliliters, about 0.1 milliliters to about 0.3 milliliters, about 0.1 milliliters to about 0.2 milliliters, about 0.2 milliliters to about 2 milliliters, about 0.3 milliliters to about 2 milliliters, about 0.4 milliliters to about 2 milliliters, about 0.5 milliliters to about 2 milliliters, about 0.6 milliliters to about 2 milliliters, about 0.7 milliliters to about 2 milliliters, about 0.8 milliliters to about 2 milliliters, about 0.9 milliliters to about 2 milliliters, about 1.0 milliliters to about 2.0 milliliters, about 1.1 milliliters to about 2.0 milliliters, about 1.2 milliliters to about 2.0 milliliters, about 1.3 milliliters to about 2.0 milliliters, about 1.4 milliliters to about 2.0 milliliters, about 1.5 milliliters to about 2.0 milliliters, about 1.6 milliliters to about 2.0 milliliters, about 1.7 to about 2.0 milliliters, about 1.8 milliliters to about 2.0 milliliters, about 1.9 milliliters to about 2.0 milliliters, or a value within these ranges. Specific examples may include about 0.1 milliliters, about 0.2 milliliters, about 0.3 milliliters, about 0.4 milliliters, about 0.5 milliliters, about 0.6 milliliters, about 0.7 milliliters, about 0.8 milliliters, about 0.9 milliliters, about 1.0 milliliters, about 1.1 milliliters, about 1.2 milliliters, about 1.3 milliliters, about 1.4 milliliters, about 1.5 milliliters, about 1.6 milliliters, about 1.7 milliliters, about 1.8 milliliters, about 1.9 milliliters, about 2.0 milliliters, or a range between any two of these values.

In some embodiment, the system for delivering a SAE composition to a subject, wherein the SAE is shelf stable. In some embodiments, the system for delivering a SAE composition to a subject, wherein the SAE composition is stable at room temperature. In some embodiments, the system for delivering a SAE composition to a subject, wherein the SABDE composition is stable at a temperature of about 23 degrees Celsius. In some embodiments, the SAE compositions described herein are stable at a temperature range of about 20 degrees Celsius to about 23 degrees Celsius. In some embodiments, the SAE compositions described herein are stable at a temperature range of about 20 degrees Celsius to about 25 degrees Celsius. In some embodiments, the SAE compositions described herein are stable at a temperature range of about 25 degrees Celsius to about 50 degrees Celsius. In some embodiments, the SAE compositions described herein are stable at a temperature range of about 5 degrees Celsius to about 50 degrees Celsius, about 5 degrees Celsius to about 40 degrees Celsius, about 5 degrees Celsius to about 30 degrees Celsius, about 5 degrees Celsius to about 28 degrees Celsius, about 5 degrees Celsius to about 26 degrees Celsius, about 5 degrees Celsius to about 25 degrees Celsius, about 5 degrees Celsius to about 24 degrees Celsius, about 5 degrees Celsius to about 22 degrees Celsius, about 5 degrees Celsius to about 20 degrees Celsius, about 5 degrees Celsius to about 18 degrees Celsius, about 5 degrees Celsius to about 16 degrees Celsius, about 5 degrees Celsius to about 14 degrees Celsius, about 5 degrees Celsius to about 12 degrees Celsius, about 5 degrees Celsius to about 10 degrees Celsius, about 5 degrees Celsius to about 8 degrees Celsius, about 5 degrees Celsius to about 6 degrees Celsius, or a value within these ranges. Specific examples may include about 5 degrees Celsius, about 10 degrees Celsius, about 15 degrees Celsius, about 20 degrees Celsius, about 21 degrees Celsius, about 22 degrees Celsius, about 23 degrees Celsius, about 24 degrees Celsius, about 25 degrees Celsius, about 26 degrees Celsius, about 28 degrees Celsius, about 30 degrees Celsius, about 35 degrees Celsius, about 40 degrees Celsius, about 45 degrees Celsius, about 50 degrees Celsius, or a range between any two of these values.

In some embodiments, the system for delivering a SAE composition to a subject, wherein the SAE composition is stable at room temperature for about 12 months. In some embodiments, the system for delivering a SAE composition to a subject, wherein the SAE composition is stable at room temperature for about 6 months to about 24 months. In some embodiments, the system for delivering a SAE composition to a subject, wherein the SAE composition is stable at room temperature for about 1 month to about 60 months, about 1 month to about 57 months, about 1 month to about 54 months, about 1 month to about 51 months, about 1 month to about 48 months, about 1 month to about 45 months, about 1 month to about 42 months, about 1 month to about 39 months, about 1 month to about 36 months, about 1 month to about 33 months, about 1 month to about 30 months, about 1 month to about 27 months, about 1 month to about 24 months, about 1 month to about 21 months, about 1 month to about 18 months, about 1 month to about 15 months, about 1 month to about 12 months, about 1 month to about 11 months, about 1 month to about 10 months, about 1 month to about 9 months, about 1 month to about 8 months, about 1 month to about 7 months, about 1 month to about 6 months, about 1 month to about 5 months, about 1 month to about 4 months, about 1 month to about 3 months, about 1 month to about 2 months, or a value within these ranges. Specific examples may include about 1 month, about 2 months, about 3 months, about 4 months, about 5 months, about 6 months, about 7 months, about 8 months, about 9 months, about 10 months, about 11 months, about 12 months, about 15 months, about 18 months, about 21 months, about 24 months, about 27 months, about 30 months, about 33 months, about 36 months, about 39 months, about 42 months, about 45 months, about 48 months, about 51 months, about 54 months, about 57 months, about 60 months, or a range between any two of these values.

In some embodiments, the system for delivering a SAE composition to a subject, wherein the SAE composition is shelf stable for about 12 months. In some embodiments, the system for delivering a SAE composition to a subject, wherein the SAE composition is shelf stable for about 6 months to about 24 months. In some embodiments, the system for delivering a SAE composition to a subject, wherein the SAE composition is shelf stable for about 1 month to about 60 months, about 1 month to about 57 months, about 1 month to about 54 months, about 1 month to about 51 months, about 1 month to about 48 months, about 1 month to about 45 months, about 1 month to about 42 months, about 1 month to about 39 months, about 1 month to about 36 months, about 1 month to about 33 months, about 1 month to about 30 months, about 1 month to about 27 months, about 1 month to about 24 months, about 1 month to about 21 months, about 1 month to about 18 months, about 1 month to about 15 months, about 1 month to about 12 months, about 1 month to about 11 months, about 1 month to about 10 months, about 1 month to about 9 months, about 1 month to about 8 months, about 1 month to about 7 months, about 1 month to about 6 months, about 1 month to about 5 months, about 1 month to about 4 months, about 1 month to about 3 months, about 1 month to about 2 months, or a value within these ranges. Specific examples may include about 1 month, about 2 months, about 3 months, about 4 months, about 5 months, about 6 months, about 7 months, about 8 months, about 9 months, about 10 months, about 11 months, about 12 months, about 15 months, about 18 months, about 21 months, about 24 months, about 27 months, about 30 months, about 33 months, about 36 months, about 39 months, about 42 months, about 45 months, about 48 months, about 51 months, about 54 months, about 57 months, about 60 months, or a range between any two of these values.

In some embodiments, the system for delivering a SAE composition to a subject comprises an applicator unit. In some embodiments, the system for delivering a SAE composition to a subject comprises an applicator unit wherein the applicator unit comprises a storage compartment, an air tight seal, a connecting region, and an applicator. In some embodiments, the storage compartment holds the SAE composition. In some embodiments, the storage unit comprises an airtight seal on the end with the connecting region. In some embodiments, the applicator unit comprises an absorbent membrane located at the distal end of the storage unit. In some embodiments, the applicator unit comprises an absorbent membrane, wherein the absorbent membrane is soaked with the SAE composition after the air tight seal is broken on the storage compartment.

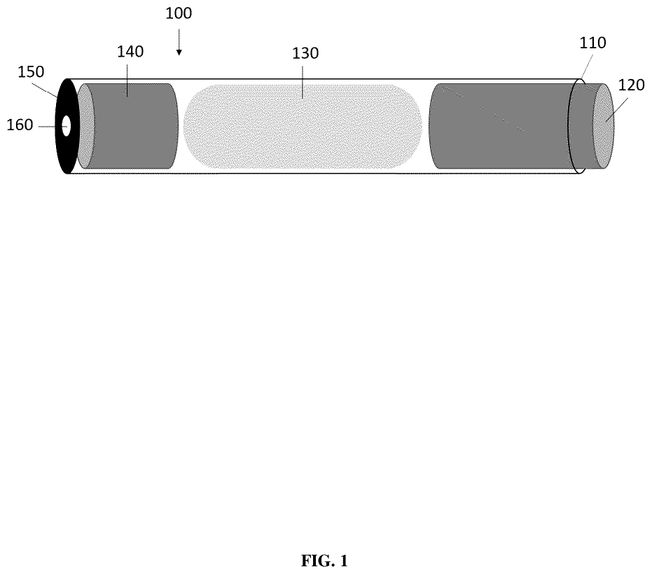

FIG. 1 is a schematic representation of an embodiment of a system for delivering a squaric acid ester (SAE) composition to a subject. The system comprises an applicator to store and apply SAE. The applicator 100 comprises an open proximal end of the applicator 110, an applicator tip 120, a glass ampule 130 containing the composition, a distal filter 140, a closed distal end of the applicator 150, and a vent hole 160 located in the closed distal end of the applicator 150. To apply the composition to subject in need thereof, the glass ampule 130 is broken by applying pressure to the applicator 100 and the composition saturates the applicator tip 120. The applicator tip 120 contacts a subject's skin to apply the composition. The distal filter 140 prevents backwards flow of glass and the composition through the vent hole 160 at the distal end of the applicator after the ampule is broken.

In some embodiments, the applicator for delivery of a SAE composition comprises an applicator tip. In some embodiments, the applicator tip is a filter. In some embodiments, the applicator tip is polyester; laminated foamed plastic; cotton; porous polyethylene; wool; gauze or other similar absorbent materials; polyolefins, nylons, polycarbonates, poly(ether sulfones), and mixtures thereof; as well as fluoropolymers, such as polyvinylidene difluoride (PVDF) and polytetrafluoroethylene (PTFE).

In some embodiments, the applicator for delivery of a SAE composition comprises a glass ampule for storage of the SAE composition. In some embodiments, the glass ampule is borosilicate glass. In some embodiments, the glass ampule protects the SAE composition from variations in temperature. In some embodiments, the glass ampule protects the SAE composition from light. In some embodiments, the glass ampule protects the SAE composition from atmospheric oxygen. In some embodiments, the SAE composition in the glass ampule is shelf stable.

In some embodiments, the applicator for delivery of a SAE composition comprises a distal filter. In some embodiments, the distal filter is a material selected from porous polyethylene; wool; gauze or other similar absorbent materials; polyolefins, nylons, polycarbonates, poly(ether sulfones), and mixtures thereof; as well as fluoropolymers, such as polyvinylidene difluoride (PVDF) and polytetrafluoroethylene (PTFE).

In some embodiments, the applicator for delivery of a SAE composition comprises a vent hole.

In some embodiments, the system for delivering a SAE composition to a subject comprises a storage compartment wherein the storage compartment protects the SAE composition from atmospheric water. In some embodiments, the system for delivering a SAE composition to a subject, wherein the SAE composition is essentially free of water. In some embodiments, the system for delivering a SAE composition to a subject, wherein the SAE composition is free of water. In some embodiments, the system for delivering a SAE composition to a subject, wherein the SAE composition is essentially free of water and DMSO.

In some embodiments, the system for delivering a SAE composition to a subject, wherein said system provides for controlled delivery of the SAE composition to the subject. In some embodiments, the system for delivering a SAE composition to a subject distributes the SAE composition evenly across an area of skin to be treated. In some embodiments, the area of skin to be treated is variable in size. In some embodiments, the maximum area of skin to be treated is about 800 cm². In some embodiments, the controlled delivery of the SAE composition to a subject, wherein said delivery provides a precise volume of the SAE composition. In some embodiments, the controlled delivery of the SAE composition to a subject, wherein said delivery is performed at a predetermined rate. In some embodiments, the controlled delivery of the SAE composition to a subject, wherein said delivery protects the SAE composition from water prior to administration to the subject.

In some embodiments, the system for delivering a SAE composition to a subject, wherein the SAE composition is administered to a subject in need thereof. In some embodiments, the system for delivering a SAE composition to a subject, wherein the SAE composition is administered topically to a subject in need thereof. In some embodiments, the system for delivering a SAE composition to a subject, wherein the SAE composition is administered to the skin of a subject in need thereof. In some embodiments, the system for delivering a SAE composition to a subject, wherein the SAE composition is administered to the scalp of a subject in need thereof. In some embodiments, the system for delivering a SAE composition to a subject, wherein the SAE composition is administered on a skin lesion of a patient in need thereof to treat a skin condition. In some embodiments, the system for delivering a SAE composition to a subject, wherein the SAE composition is administered adjacent to a skin lesion of a patient in need thereof to treat a skin condition. In some embodiments, the system for delivering a SAE composition to a subject, wherein the SAE composition is administered to an area of skin that is determined to be optimal by a physician. In some embodiments, the system for delivering a SAE composition to a subject, wherein the SAE composition is administered selectively to an area of skin in need thereof. In some embodiments, the system for delivering a SAE composition to a subject, wherein the SAE composition does not make contact with any unintended area of skin. In some embodiments, the system for delivering a SAE composition to a subject, wherein the SAE composition is entirely contained within the storage unit and applicator unit.

In some embodiments, the system for delivering a SAE composition to a subject with a skin disease. In some embodiments, the system for delivering a SAE composition to a subject with hair loss. In some embodiments, the system for delivering a SAE composition to a subject with alopecia areata. In some embodiments, the system for delivering a SAE composition to a subject to treat a condition selected from viral warts, herpes simplex virus, molluscum contagiousum, and cutaneous t cell lymphoma.

In some embodiments, the system for delivering a SAE composition to a subject in a therapeutically effective amount. In some embodiments, the therapeutically effective amount is an amount disclosed herein. In some embodiments, the system for delivering a SAE composition to a subject leads to a disappearance of the conditions being treated. In some embodiments, the system for delivering a SAE composition to a subject leads to restoration of hair loss. In some embodiments, the system for delivering a SAE composition to a subject leads to hair growth on regions of skin in direct contact with the SAE composition. In some embodiments, the system for delivering a SAE composition to a subject leads to hair growth on regions of skin adjacent to the location of administration of the SAE composition.

In some embodiments, the subject is diagnosed with alopecia areata and successful treatment of this condition results in a one point improvement on any ordinal or interval assessment scale, such as the Physician Global Assessment or the Investigator Global Assessment. In some embodiments, the subject is diagnosed with alopecia areata and successful treatment of this condition results in about 100% restoration of hair growth in the location of the patch is administered. In some embodiments, the subject is diagnosed with alopecia areata and successful treatment of this condition results in at least about 50% increase in hair growth as defined by number of hair follicles with actively growing hair shafts as seen with clinical, trichoscopic, or histopathological assessment in the location of the patch is administered. In some embodiments, successful treatment of alopecia areata with the SAE composition results in an increase in hair growth in the location the composition is administered by a range from about 50% to about 100%, about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 60% to about 100%, about 60% to about 95%, about 60% to about 90%, about 60% to about 85%, about 60% to about 80%, about 60% to about 75%, about 60% to about 70%, about 60% to about 65%, about 70% to about 100%, about 70% to about 95%, about 70% to about 90%, about 70% to about 85%, about 70% to about 80%, about 70% to about 75%, about 80% to about 100%, about 80% to about 95%, about 80% to about 90%, about 80% to about 85%, about 90% to about 100%, about 90% to about 95%, or a value within these ranges. Specific examples may include increasing hair growth as defined by the number of hair follicles with actively growing hair shafts as seen with clinical, trichoscopic, or histopathologic assessment, by about 100%, about 99%, about 95%, about 90%, about 85%, about 80%, about 75%, about 70%, about 65%, about 60%, about 55%, about 50% or a range between any two of these values.

In some embodiments, successful treatment of alopecia areata with the SAE composition results in improvement in the Severity Alopecia Tool (SALT) score in the location the patch is administered by a range from about 50% to about 100%, about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 60% to about 100%, about 60% to about 95%, about 60% to about 90%, about 60% to about 85%, about 60% to about 80%, about 60% to about 75%, about 60% to about 70%, about 60% to about 65%, about 70% to about 100%, about 70% to about 95%, about 70% to about 90%, about 70% to about 85%, about 70% to about 80%, about 70% to about 75%, about 80% to about 100%, about 80% to about 95%, about 80% to about 90%, about 80% to about 85%, about 90% to about 100%, about 90% to about 95%, or a value within these ranges. Specific examples may include improving the Severity Alopecia Tool (SALT) score by about 100%, about 99%, about 95%, about 90%, about 85%, about 80%, about 75%, about 70%, about 65%, about 60%, about 55%, about 50% or a range between any two of these values.

In some embodiments, the system for delivering a SAE composition to a subject, wherein the SAE composition is delivered to the subject once per month. In some embodiments, the system for delivering a SAE composition to a subject, wherein the SAE composition is delivered to the subject once per month for four months. In some embodiments, the system for delivering a SAE composition to a subject, wherein the SAE composition is delivered to the subject once per month for one to 12 months, for one to 11 months, for one to 10 months, for one to nine months, for one to eight months, for one to seven months, for one to six months, for one to five months, for one to four months, for one to three months, for one to two months, a value within these ranges. Specific examples may include one month, two months, three months, four months, five months, six months, seven months, eight months, nine months, 10 months, 11 months, 12 months, or a range between any two of these values.

In some embodiments, the system for delivering a SAE composition to a subject, wherein the subject is evaluated at the conclusion of four months of treatment for hair regrowth. In some embodiments, the system for delivering a SAE composition to a subject, wherein the subject is evaluated for hair regrowth at the conclusion of four months of treatment. Subjects that experience hair regrowth at the conclusion of four months of treatment are referred to as “responders.” Subjects that do not experience hair regrowth at the conclusion of four months of treatment are referred to as “non-responders.” Subjects that experience measurable hair regrowth at any amount less than about a 50% SALT score at the conclusion of four months of treatment are referred to as “partial responders.” In some embodiments, the system for delivering a SAE composition to a subject, wherein the subject is a responder. In some embodiments, the system for delivering a SAE composition to a subject, wherein the subject is a non-responder. In some embodiments, the system for delivering a SAE composition to a subject, wherein the subject is a partial responder. In some embodiments, the system for delivering a SAE composition to a non-responder, wherein the SAE composition is delivered every two weeks. In some embodiments, the system for delivering a SAE composition to a partial responder, wherein the SAE composition is delivered every two weeks. In some embodiments, the system for delivering a SAE composition to non-responder or a partial responder, wherein the SAE composition is delivered every week. In some embodiments, the system for delivering a SAE composition to a non-responder or a partial responder, wherein the SAE composition is delivered twice per week. In some embodiments, the system for delivering a SAE composition to a non-responder or a partial responder, wherein the SAE composition is delivered every 3 weeks.

### A Method of Delivering a SABDE Composition

Embodiments of the present application are directed to a method of delivering a squaric acid ester (SAE) composition to a subject for treating a condition susceptible to treatment with the SAE composition, comprising: providing applicator device comprising a storage compartment, an air tight seal, a connecting region, and an applicator; and delivering the SAE composition from the storage compartment through the applicator to the subject.

In some embodiments, the SAE composition comprises squaric acid dibutylester (SADBE). In some embodiments, the SAE composition comprises squaric acid diethylester (SADEE). In some embodiments, the SAE composition comprises a single SAE. In some embodiments, the SAE composition comprises a combination of SAEs. In some embodiments, the SEA composition comprises SADBE and SADEE.

In some embodiments, the method for delivering a SAE composition to a subject, wherein the SAE composition comprises at least about 2.5% (w/v) SAE. In some embodiments, the method for delivering a SAE composition to a subject, wherein the SAE composition comprises at least about 5% (w/v) SAE. In some embodiments, the method for delivering a SAE composition to a subject, wherein the SAE composition comprises at least about 7% (w/v) SAE. In some embodiments, the method for delivering a SAE composition to a subject, wherein the SAE composition comprises at least about 10% (w/v) SAE. In some embodiments, the method for delivering a SAE composition to a subject, wherein the SAE composition comprises a range of SAE from about 1% to about 20%, about 1% to about 18%, about 10% to about 16%, about 1% to about 15%, about 1% to about 14%, about 1% to about 12%, about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 20%, about 3% to about 20%, about 4% to about 20%, about 5% to about 20%, about 6% to about 20%, about 7% to about 20%, about 8% to about 20%, about 9% to about 20%, about 10% to about 20%, about 12% to about 20%, about 14% to about 20%, about 15% to about 20%, about 16% to about 20%, about 18% to about 20%, or a value within these ranges. Specific examples may include about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20% or a range between any two of these values.

Some embodiments disclosed herein also include pharmaceutical compositions which contain, as the active ingredient, one or more of the compounds disclosed herein in combination with one or more pharmaceutically acceptable carriers (excipients). In some embodiments, the excipient may be selected from at least one of the following: ketones, alcohols, other organic solvents.

In some embodiments, the method for delivering a SAE composition to a subject comprises delivering a therapeutically effective volume of the SAE composition. In some embodiments, the method for delivering a SAE composition to a subject comprises delivering an effective volume of the SAE composition. In some embodiments, the method for delivering a SAE composition to a subject comprises delivering about 1 milliliter of the SAE composition. In some embodiments, the method for delivering a SAE composition to a subject comprises delivering about 0.8 milliliters of the SAE composition. In some embodiments, the method for delivering a SAE composition to a subject comprises delivering about 1.2 milliliters of the SAE composition. In some embodiments, the method for delivering a SAE composition to a subject comprises delivering a range of about 0.7 milliliters to about 1.3 milliliters of the SAE composition. In some embodiments, the method for delivering a SAE composition to a subject comprises delivering a range of about 0.1 milliliters to about 2 milliliters, about 0.1 milliliters to about 1.9 milliliters, about 0.1 milliliters to about 1.8 milliliters, about 0.1 milliliters to about 1.7 milliliters, about 0.1 milliliters to about 1.6 milliliters, about 0.1 to about 1.5 milliliters, about 0.1 milliliters to about 1.4 milliliters, about 0.1 milliliters to about 1.3 milliliters, about 0.1 milliliters to about 1.2 milliliters, about 0.1 milliliters to about 1.1 milliliters, about 0.1 milliliters to about 1.0 milliliters, about 0.1 milliliters to about 0.9 milliliters, about 0.1 milliliters to about 0.8 milliliters, about 0.1 milliliters to about 0.7 milliliters, about 0.1 milliliters to about 0.6 milliliters, about 0.1 milliliters to about 0.5 milliliters, about 0.1 milliliters to about 0.4 milliliters, about 0.1 milliliters to about 0.3 milliliters, about 0.1 milliliters to about 0.2 milliliters, about 0.2 milliliters to about 2 milliliters, about 0.3 milliliters to about 2 milliliters, about 0.4 milliliters to about 2 milliliters, about 0.5 milliliters to about 2 milliliters, about 0.6 milliliters to about 2 milliliters, about 0.7 milliliters to about 2 milliliters, about 0.8 milliliters to about 2 milliliters, about 0.9 milliliters to about 2 milliliters, about 1.0 milliliters to about 2.0 milliliters, about 1.1 milliliters to about 2.0 milliliters, about 1.2 milliliters to about 2.0 milliliters, about 1.3 milliliters to about 2.0 milliliters, about 1.4 milliliters to about 2.0 milliliters, about 1.5 milliliters to about 2.0 milliliters, about 1.6 milliliters to about 2.0 milliliters, about 1.7 to about 2.0 milliliters, about 1.8 milliliters to about 2.0 milliliters, about 1.9 milliliters to about 2.0 milliliters, or a value within these ranges. Specific examples may include about 0.1 milliliters, about 0.2 milliliters, about 0.3 milliliters, about 0.4 milliliters, about 0.5 milliliters, about 0.6 milliliters, about 0.7 milliliters, about 0.8 milliliters, about 0.9 milliliters, about 1.0 milliliters, about 1.1 milliliters, about 1.2 milliliters, about 1.3 milliliters, about 1.4 milliliters, about 1.5 milliliters, about 1.6 milliliters, about 1.7 milliliters, about 1.8 milliliters, about 1.9 milliliters, about 2.0 milliliters, or a range between any two of these values.

In some embodiment, the method for delivering a SAE composition to a subject, wherein the SAE is shelf stable. In some embodiments, the method for delivering a SAE composition to a subject, wherein the SAE composition is stable at room temperature. In some embodiments, the method for delivering a SAE composition to a subject, wherein the SABDE composition is stable at a temperature of about 23 degrees Celsius. In some embodiments, the method for delivering a SAE composition to a subject, wherein the SAE composition is stable at a temperature range of about 20 degrees Celsius to about 23 degrees Celsius. In some embodiments, the method for delivering a SAE composition to a subject, wherein the SAE composition is stable at a temperature range of about 20 degrees Celsius to about 25 degrees Celsius. In some embodiments, the method for delivering a SAE composition to a subject, wherein the SAE composition is stable at a temperature range of about 25 degrees Celsius to about 50 degrees Celsius. In some embodiments, the method for delivering a SAE composition to a subject, wherein the SAE composition is stable at a temperature range of about 5 degrees Celsius to about 50 degrees Celsius, about 5 degrees Celsius to about 40 degrees Celsius, about 5 degrees Celsius to about 30 degrees Celsius, about 5 degrees Celsius to about 28 degrees Celsius, about 5 degrees Celsius to about 26 degrees Celsius, about 5 degrees Celsius to about 25 degrees Celsius, about 5 degrees Celsius to about 24 degrees Celsius, about 5 degrees Celsius to about 22 degrees Celsius, about 5 degrees Celsius to about 20 degrees Celsius, about 5 degrees Celsius to about 18 degrees Celsius, about 5 degrees Celsius to about 16 degrees Celsius, about 5 degrees Celsius to about 14 degrees Celsius, about 5 degrees Celsius to about 12 degrees Celsius, about 5 degrees Celsius to about 10 degrees Celsius, about 5 degrees Celsius to about 8 degrees Celsius, about 5 degrees Celsius to about 6 degrees Celsius, or a value within these ranges. Specific examples may include about 5 degrees Celsius, about 10 degrees Celsius, about 15 degrees Celsius, about 20 degrees Celsius, about 21 degrees Celsius, about 22 degrees Celsius, about 23 degrees Celsius, about 24 degrees Celsius, about 25 degrees Celsius, about 26 degrees Celsius, about 28 degrees Celsius, about 30 degrees Celsius, about 35 degrees Celsius, about 40 degrees Celsius, about 45 degrees Celsius, about 50 degrees Celsius, or a range between any two of these values.

In some embodiments, the method for delivering a SAE composition to a subject, wherein the SAE composition is stable at room temperature for about 12 months. In some embodiments, the method for delivering a SAE composition to a subject, wherein the SAE composition is stable at room temperature for about 6 months to about 24 months. In some embodiments, the method for delivering a SAE composition to a subject, wherein the SAE composition is stable at room temperature for about 1 month to about 60 months, about 1 month to about 57 months, about 1 month to about 54 months, about 1 month to about 51 months, about 1 month to about 48 months, about 1 month to about 45 months, about 1 month to about 42 months, about 1 month to about 39 months, about 1 month to about 36 months, about 1 month to about 33 months, about 1 month to about 30 months, about 1 month to about 27 months, about 1 month to about 24 months, about 1 month to about 21 months, about 1 month to about 18 months, about 1 month to about 15 months, about 1 month to about 12 months, about 1 month to about 11 months, about 1 month to about 10 months, about 1 month to about 9 months, about 1 month to about 8 months, about 1 month to about 7 months, about 1 month to about 6 months, about 1 month to about 5 months, about 1 month to about 4 months, about 1 month to about 3 months, about 1 month to about 2 months, or a value within these ranges. Specific examples may include about 1 month, about 2 months, about 3 months, about 4 months, about 5 months, about 6 months, about 7 months, about 8 months, about 9 months, about 10 months, about 11 months, about 12 months, about 15 months, about 18 months, about 21 months, about 24 months, about 27 months, about 30 months, about 33 months, about 36 months, about 39 months, about 42 months, about 45 months, about 48 months, about 51 months, about 54 months, about 57 months, about 60 months, or a range between any two of these values.

In some embodiments, the method for delivering a SAE composition to a subject, wherein the SAE composition is shelf stable for about 12 months. In some embodiments, the method for delivering a SAE composition to a subject, wherein the SAE composition is shelf stable for about 6 months to about 24 months. In some embodiments, the method for delivering a SAE composition to a subject, wherein the SAE composition is shelf stable for about 1 month to about 60 months, about 1 month to about 57 months, about 1 month to about 54 months, about 1 month to about 51 months, about 1 month to about 48 months, about 1 month to about 45 months, about 1 month to about 42 months, about 1 month to about 39 months, about 1 month to about 36 months, about 1 month to about 33 months, about 1 month to about 30 months, about 1 month to about 27 months, about 1 month to about 24 months, about 1 month to about 21 months, about 1 month to about 18 months, about 1 month to about 15 months, about 1 month to about 12 months, about 1 month to about 11 months, about 1 month to about 10 months, about 1 month to about 9 months, about 1 month to about 8 months, about 1 month to about 7 months, about 1 month to about 6 months, about 1 month to about 5 months, about 1 month to about 4 months, about 1 month to about 3 months, about 1 month to about 2 months, or a value within these ranges. Specific examples may include about 1 month, about 2 months, about 3 months, about 4 months, about 5 months, about 6 months, about 7 months, about 8 months, about 9 months, about 10 months, about 11 months, about 12 months, about 15 months, about 18 months, about 21 months, about 24 months, about 27 months, about 30 months, about 33 months, about 36 months, about 39 months, about 42 months, about 45 months, about 48 months, about 51 months, about 54 months, about 57 months, about 60 months, or a range between any two of these values.

In some embodiments, the method for delivering a SAE composition to a subject, wherein the method comprises an applicator unit. In some embodiments, the method for delivering a SAE composition to a subject, wherein the method comprises an applicator unit wherein the applicator unit comprises a storage compartment, an air tight seal, a connecting region, and an applicator. In some embodiments, the storage compartment holds the SAE composition. In some embodiments, the storage unit comprises an airtight seal on the end with the connecting region. In some embodiments, the applicator unit comprises an absorbent membrane located at the distal end of the storage unit. In some embodiments, the applicator unit comprises an absorbent membrane, wherein the absorbent membrane is soaked with the SAE composition after the air tight seal is broken on the storage compartment.

In some embodiments, the method for delivering a SAE composition to a subject with an applicator unit, wherein the applicator unit comprises a storage compartment. In some embodiments, the method for delivering a SAE composition to a subject with an applicator unit, wherein the applicator unit comprises a storage compartment to protect the SAE composition from atmospheric water. In some embodiments, the method for delivering a SAE composition to a subject, wherein the SAE composition is essentially free of water. In some embodiments, the method for delivering a SAE composition to a subject, wherein the SAE composition is free of water. In some embodiments, the method for delivering a SAE composition to a subject, wherein the SAE composition is essentially free of water and DMSO.

In some embodiments, the method for delivering a SAE composition to a subject, wherein said method provides for controlled delivery of the SAE composition to the subject. In some embodiments, the method for delivering a SAE composition to a subject distributes the SAE composition evenly across an area of skin to be treated. In some embodiments, the area of skin to be treated is variable in size. In some embodiments, the maximum area of skin to be treated is about 800 cm². In some embodiments, the controlled delivery of the SAE composition to a subject, wherein said delivery provides a precise volume of the SAE composition. In some embodiments, the controlled delivery of the SAE composition to a subject, wherein said delivery is performed at a predetermined rate. In some embodiments, the controlled delivery of the SAE composition to a subject, wherein said delivery protects the SAE composition from water prior to administration to the subject.

In some embodiments, the method for delivering a SAE composition to a subject, wherein the SAE composition is administered to a subject in need thereof. In some embodiments, the method for delivering a SAE composition to a subject, wherein the SAE composition is administered topically to a subject in need thereof. In some embodiments, the method for delivering a SAE composition to a subject, wherein the SAE composition is administered to the skin of a subject in need thereof. In some embodiments, the system for delivering a SAE composition to a subject, wherein the SAE composition is administered to the scalp of a subject in need thereof. In some embodiments, the method for delivering a SAE composition to a subject, wherein the SAE composition is administered on a skin lesion of a patient in need thereof to treat a skin condition. In some embodiments, the method for delivering a SAE composition to a subject, wherein the SAE composition is administered adjacent to a skin lesion of a patient in need thereof to treat a skin condition. In some embodiments, the method for delivering a SAE composition to a subject, wherein the SAE composition is administered to an area of the skin that is determined to be optimal by a physician. In some embodiments, the method for delivering a SAE composition to a subject, wherein the SAE composition is administered selectively to an area of skin in need thereof. In some embodiments, the method for delivering a SAE composition to a subject, wherein the SAE composition does not make contact with any unintended area of skin. In some embodiments, the method for delivering a SAE composition to a subject, wherein the SAE composition is entirely contained within the storage unit and applicator unit.

In some embodiments, the system for delivering a SAE composition to a subject with a skin disease. In some embodiments, the method for delivering a SAE composition to a subject with hair loss. In some embodiments, the method for delivering a SAE composition to a subject with alopecia areata. In some embodiments, the method for delivering a SAE composition to a subject to treat a condition selected from viral warts, herpes simplex virus, molluscum contagiousum, and cutaneous t cell lymphoma.

In some embodiments, the method for delivering a SAE composition to a subject in a therapeutically effective amount. In some embodiments, the therapeutically effective amount is an amount disclosed herein. In some embodiments, the method for delivering a SAE composition to a subject leads to a disappearance of the conditions being treated. In some embodiments, the method for delivering a SAE composition to a subject leads to restoration of hair loss. In some embodiments, the method for delivering a SAE composition to a subject leads to hair growth on regions of skin in direct contact with the SAE composition. In some embodiments, the method for delivering a SAE composition to a subject leads to hair growth on regions of skin adjacent to the location of administration of the SAE composition.

In some embodiments, the subject is diagnosed with alopecia areata and successful treatment of this condition results in a one point improvement on any ordinal or interval assessment scale, such as the Physician Global Assessment or the Investigator Global Assessment. In some embodiments, the subject is diagnosed with alopecia areata and successful treatment of this condition results in about 100% restoration of hair growth in the location of the patch is administered. In some embodiments, the subject is diagnosed with alopecia areata and successful treatment of this condition results in at least about 50% increase in hair growth as defined by number of hair follicles with actively growing hair shafts as seen with clinical, trichoscopic, or histopathological assessment in the location of the patch is administered. In some embodiments, successful treatment of alopecia areata with the SAE composition results in an increase in hair growth in the location the composition is administered by a range from about 50% to about 100%, about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 60% to about 100%, about 60% to about 95%, about 60% to about 90%, about 60% to about 85%, about 60% to about 80%, about 60% to about 75%, about 60% to about 70%, about 60% to about 65%, about 70% to about 100%, about 70% to about 95%, about 70% to about 90%, about 70% to about 85%, about 70% to about 80%, about 70% to about 75%, about 80% to about 100%, about 80% to about 95%, about 80% to about 90%, about 80% to about 85%, about 90% to about 100%, about 90% to about 95%, or a value within these ranges. Specific examples may include increasing hair growth as defined by the number of hair follicles with actively growing hair shafts as seen with clinical, trichoscopic, or histopathologic assessment, by about 100%, about 99%, about 95%, about 90%, about 85%, about 80%, about 75%, about 70%, about 65%, about 60%, about 55%, about 50% or a range between any two of these values.

In some embodiments, successful treatment of alopecia areata with the SAE composition results in improvement in the Severity Alopecia Tool (SALT) score in the location the patch is administered by a range from about 50% to about 100%, about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 60% to about 100%, about 60% to about 95%, about 60% to about 90%, about 60% to about 85%, about 60% to about 80%, about 60% to about 75%, about 60% to about 70%, about 60% to about 65%, about 70% to about 100%, about 70% to about 95%, about 70% to about 90%, about 70% to about 85%, about 70% to about 80%, about 70% to about 75%, about 80% to about 100%, about 80% to about 95%, about 80% to about 90%, about 80% to about 85%, about 90% to about 100%, about 90% to about 95%, or a value within these ranges. Specific examples may include improving the Severity Alopecia Tool (SALT) score by about 100%, about 99%, about 95%, about 90%, about 85%, about 80%, about 75%, about 70%, about 65%, about 60%, about 55%, about 50% or a range between any two of these values.

In some embodiments, the method for delivering a SAE composition to a subject, wherein the SAE composition is delivered to the subject once per month. In some embodiments, the method for delivering a SAE composition to a subject, wherein the SAE composition is delivered to the subject once per month for four months. In some embodiments, the method for delivering a SAE composition to a subject, wherein the SAE composition is delivered to the subject once per month for one to 12 months, for one to 11 months, for one to 10 months, for one to nine months, for one to eight months, for one to seven months, for one to six months, for one to five months, for one to four months, for one to three months, for one to two months, a value within these ranges. Specific examples may include one month, two months, three months, four months, five months, six months, seven months, eight months, nine months, 10 months, 11 months, 12 months, or a range between any two of these values.

In some embodiments, the method for delivering a SAE composition to a subject, wherein the subject is evaluated at the conclusion of four months of treatment for hair regrowth. In some embodiments, the method for delivering a SAE composition to a subject, wherein the subject is evaluated for hair regrowth at the conclusion of four months of treatment. Subjects that experience hair regrowth at the conclusion of four months of treatment are referred to as “responders.” Subjects that do not experience hair regrowth at the conclusion of four months of treatment are referred to as “non-responders.” Subjects that experience measurable hair regrowth at any amount less than about a 50% SALT score at the conclusion of four months of treatment are referred to as “partial responders.” In some embodiments, the method for delivering a SAE composition to a subject, wherein the subject is a responder. In some embodiments, the method for delivering a SAE composition to a subject, wherein the subject is a non-responder. In some embodiments, the method for delivering a SAE composition to a subject, wherein the subject is a partial responder. In some embodiments, the method for delivering a SAE composition to a non-responder, wherein the SAE composition is delivered every two weeks. In some embodiments, the method for delivering a SAE composition to a partial responder, wherein the SAE composition is delivered every two weeks. In some embodiments, the method for delivering a SAE composition to non-responder or a partial responder, wherein the SAE composition is delivered every week. In some embodiments, the method for delivering a SAE composition to a non-responder or a partial responder, wherein the SAE composition is delivered twice per week. In some embodiments, the method for delivering a SAE composition to a non-responder or a partial responder, wherein the SAE composition is delivered every 3 weeks.

### Kit for Delivering a SAE Composition

Embodiments of the present application are directed to a kit for administering a squaric acid ester (SAE) composition to a subject, comprising: an applicator device comprising a storage compartment, an air tight seal, a connecting region, and an applicator; the SAE composition comprising at least 5% SAE; said kit further comprising instructional material for administering said SAE composition for treating a condition susceptible to treatment using said SAE composition.

In some embodiments, the SAE composition comprises squaric acid dibutylester (SADBE). In some embodiments, the SAE composition comprises squaric acid diethylester (SADEE). In some embodiments, the SAE composition comprises a single SAE. In some embodiments, the SAE composition comprises a combination of SAEs. In some embodiments, the SEA composition comprises SADBE and SADEE.

In some embodiments, the kit for delivering a SAE composition to a subject, wherein the SAE composition comprises at least about 2.5% (w/v) SAE. In some embodiments, the method for delivering a SAE composition to a subject, wherein the SAE composition comprises at least about 5% (w/v) SAE. In some embodiments, the kit for delivering a SAE composition to a subject, wherein the SAE composition comprises at least about 7% (w/v) SAE. In some embodiments, the kit for delivering a SAE composition to a subject, wherein the SAE composition comprises at least about 10% (w/v) SAE. In some embodiments, the kit for delivering a SAE composition to a subject, wherein the SAE composition comprises a range of SAE from about 1% to about 20%, about 1% to about 18%, about 1% to about 16%, about 1% to about 15%, about 1% to about 14%, about 1% to about 12%, about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 2% to about 20%, about 3% to about 20%, about 4% to about 20%, about 5% to about 20%, about 6% to about 20%, about 7% to about 20%, about 8% to about 20%, about 9% to about 20%, about 10% to about 20%, about 12% to about 20%, about 14% to about 20%, about 15% to about 20%, about 16% to about 20%, about 18% to about 20%, or a value within these ranges. Specific examples may include about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20% or a range between any two of these values.

Some embodiments disclosed herein also include pharmaceutical compositions which contain, as the active ingredient, one or more of the compounds disclosed herein in combination with one or more pharmaceutically acceptable carriers (excipients). In some embodiments, the excipient may be selected from at least one of the following: ketones, alcohols, other organic solvents.

In some embodiments, the kit for delivering a SAE composition to a subject comprises delivering a therapeutically effective volume of the SAE composition. In some embodiments, the kit for delivering a SAE composition to a subject comprises delivering an effective volume of the SAE composition. In some embodiments, the kit for delivering a SAE composition to a subject comprises delivering about 1 milliliter of the SAE composition. In some embodiments, the kit for delivering a SAE composition to a subject comprises delivering about 0.8 milliliters of the SAE composition. In some embodiments, the kit for delivering a SAE composition to a subject comprises delivering about 1.2 milliliters of the SAE composition. In some embodiments, the kit for delivering a SAE composition to a subject comprises delivering a range of about 0.7 milliliters to about 1.3 milliliters of the SAE composition. In some embodiments, the kit for delivering a SAE composition to a subject comprises delivering a range of about 0.1 milliliters to about 2 milliliters, about 0.1 milliliters to about 1.9 milliliters, about 0.1 milliliters to about 1.8 milliliters, about 0.1 milliliters to about 1.7 milliliters, about 0.1 milliliters to about 1.6 milliliters, about 0.1 to about 1.5 milliliters, about 0.1 milliliters to about 1.4 milliliters, about 0.1 milliliters to about 1.3 milliliters, about 0.1 milliliters to about 1.2 milliliters, about 0.1 milliliters to about 1.1 milliliters, about 0.1 milliliters to about 1.0 milliliters, about 0.1 milliliters to about 0.9 milliliters, about 0.1 milliliters to about 0.8 milliliters, about 0.1 milliliters to about 0.7 milliliters, about 0.1 milliliters to about 0.6 milliliters, about 0.1 milliliters to about 0.5 milliliters, about 0.1 milliliters to about 0.4 milliliters, about 0.1 milliliters to about 0.3 milliliters, about 0.1 milliliters to about 0.2 milliliters, about 0.2 milliliters to about 2 milliliters, about 0.3 milliliters to about 2 milliliters, about 0.4 milliliters to about 2 milliliters, about 0.5 milliliters to about 2 milliliters, about 0.6 milliliters to about 2 milliliters, about 0.7 milliliters to about 2 milliliters, about 0.8 milliliters to about 2 milliliters, about 0.9 milliliters to about 2 milliliters, about 1.0 milliliters to about 2.0 milliliters, about 1.1 milliliters to about 2.0 milliliters, about 1.2 milliliters to about 2.0 milliliters, about 1.3 milliliters to about 2.0 milliliters, about 1.4 milliliters to about 2.0 milliliters, about 1.5 milliliters to about 2.0 milliliters, about 1.6 milliliters to about 2.0 milliliters, about 1.7 to about 2.0 milliliters, about 1.8 milliliters to about 2.0 milliliters, about 1.9 milliliters to about 2.0 milliliters, or a value within these ranges. Specific examples may include about 0.1 milliliters, about 0.2 milliliters, about 0.3 milliliters, about 0.4 milliliters, about 0.5 milliliters, about 0.6 milliliters, about 0.7 milliliters, about 0.8 milliliters, about 0.9 milliliters, about 1.0 milliliters, about 1.1 milliliters, about 1.2 milliliters, about 1.3 milliliters, about 1.4 milliliters, about 1.5 milliliters, about 1.6 milliliters, about 1.7 milliliters, about 1.8 milliliters, about 1.9 milliliters, about 2.0 milliliters, or a range between any two of these values.

In some embodiment, the kit for delivering a SAE composition to a subject, wherein the SAE is shelf stable. In some embodiments, the kit for delivering a SAE composition to a subject, wherein the SAE composition is stable at room temperature. In some embodiments, the kit for delivering a SAE composition to a subject, wherein the SABDE composition is stable at a temperature of about 23 degrees Celsius. In some embodiments, the kit for delivering a SAE composition to a subject, wherein the SAE composition is stable at a temperature range of about 20 degrees Celsius to about 23 degrees Celsius. In some embodiments, the kit for delivering a SAE composition to a subject, wherein the SAE composition is stable at a temperature range of about 20 degrees Celsius to about 25 degrees Celsius. In some embodiments, the kit for delivering a SAE composition to a subject, where in the SAE composition is stable at a temperature range of about 25 degrees Celsius to about 50 degrees Celsius. In some embodiments, the kit for delivering a SAE composition to a subject, wherein the SAE composition is stable at a temperature range of about 5 degrees Celsius to about 50 degrees Celsius, about 5 degrees Celsius to about 40 degrees Celsius, about 5 degrees Celsius to about 30 degrees Celsius, about 5 degrees Celsius to about 28 degrees Celsius, about 5 degrees Celsius to about 26 degrees Celsius, about 5 degrees Celsius to about 25 degrees Celsius, about 5 degrees Celsius to about 24 degrees Celsius, about 5 degrees Celsius to about 22 degrees Celsius, about 5 degrees Celsius to about 20 degrees Celsius, about 5 degrees Celsius to about 18 degrees Celsius, about 5 degrees Celsius to about 16 degrees Celsius, about 5 degrees Celsius to about 14 degrees Celsius, about 5 degrees Celsius to about 12 degrees Celsius, about 5 degrees Celsius to about 10 degrees Celsius, about 5 degrees Celsius to about 8 degrees Celsius, about 5 degrees Celsius to about 6 degrees Celsius, or a value within these ranges. Specific examples may include about 5 degrees Celsius, about 10 degrees Celsius, about 15 degrees Celsius, about 20 degrees Celsius, about 21 degrees Celsius, about 22 degrees Celsius, about 23 degrees Celsius, about 24 degrees Celsius, about 25 degrees Celsius, about 26 degrees Celsius, about 28 degrees Celsius, about 30 degrees Celsius, about 35 degrees Celsius, about 40 degrees Celsius, about 45 degrees Celsius, about 50 degrees Celsius, or a range between any two of these values.

In some embodiments, the kit for delivering a SAE composition to a subject, wherein the SAE composition is stable at room temperature for about 12 months. In some embodiments, the kit for delivering a SAE composition to a subject, wherein the SAE composition is stable at room temperature for about 6 months to about 24 months. In some embodiments, the kit for delivering a SAE composition to a subject, wherein the SAE composition is stable at room temperature for about 1 month to about 60 months, about 1 month to about 57 months, about 1 month to about 54 months, about 1 month to about 51 months, about 1 month to about 48 months, about 1 month to about 45 months, about 1 month to about 42 months, about 1 month to about 39 months, about 1 month to about 36 months, about 1 month to about 33 months, about 1 month to about 30 months, about 1 month to about 27 months, about 1 month to about 24 months, about 1 month to about 21 months, about 1 month to about 18 months, about 1 month to about 15 months, about 1 month to about 12 months, about 1 month to about 11 months, about 1 month to about 10 months, about 1 month to about 9 months, about 1 month to about 8 months, about 1 month to about 7 months, about 1 month to about 6 months, about 1 month to about 5 months, about 1 month to about 4 months, about 1 month to about 3 months, about 1 month to about 2 months, or a value within these ranges. Specific examples may include about 1 month, about 2 months, about 3 months, about 4 months, about 5 months, about 6 months, about 7 months, about 8 months, about 9 months, about 10 months, about 11 months, about 12 months, about 15 months, about 18 months, about 21 months, about 24 months, about 27 months, about 30 months, about 33 months, about 36 months, about 39 months, about 42 months, about 45 months, about 48 months, about 51 months, about 54 months, about 57 months, about 60 months, or a range between any two of these values.

In some embodiments, the kit for delivering a SAE composition to a subject, wherein the SAE composition is shelf stable for about 12 months. In some embodiments, the kit for delivering a SAE composition to a subject, wherein the SAE composition is shelf stable for about 6 months to about 24 months. In some embodiments, the kit for delivering a SAE composition to a subject, wherein the SAE composition is shelf stable for about 1 month to about 60 months, about 1 month to about 57 months, about 1 month to about 54 months, about 1 month to about 51 months, about 1 month to about 48 months, about 1 month to about 45 months, about 1 month to about 42 months, about 1 month to about 39 months, about 1 month to about 36 months, about 1 month to about 33 months, about 1 month to about 30 months, about 1 month to about 27 months, about 1 month to about 24 months, about 1 month to about 21 months, about 1 month to about 18 months, about 1 month to about 15 months, about 1 month to about 12 months, about 1 month to about 11 months, about 1 month to about 10 months, about 1 month to about 9 months, about 1 month to about 8 months, about 1 month to about 7 months, about 1 month to about 6 months, about 1 month to about 5 months, about 1 month to about 4 months, about 1 month to about 3 months, about 1 month to about 2 months, or a value within these ranges. Specific examples may include about 1 month, about 2 months, about 3 months, about 4 months, about 5 months, about 6 months, about 7 months, about 8 months, about 9 months, about 10 months, about 11 months, about 12 months, about 15 months, about 18 months, about 21 months, about 24 months, about 27 months, about 30 months, about 33 months, about 36 months, about 39 months, about 42 months, about 45 months, about 48 months, about 51 months, about 54 months, about 57 months, about 60 months, or a range between any two of these values.

In some embodiments, the kit for delivering a SAE composition to a subject, wherein the kit comprises an applicator unit. In some embodiments, the kit for delivering a SAE composition to a subject, wherein the kit comprises an applicator unit wherein the applicator unit comprises a storage compartment, an air tight seal, a connecting region, and an applicator. In some embodiments, the storage compartment holds the SAE composition. In some embodiments, the storage unit comprises an airtight seal on the end with the connecting region. In some embodiments, the applicator unit comprises an absorbent membrane located at the distal end of the storage unit. In some embodiments, the applicator unit comprises an absorbent membrane, wherein the absorbent membrane is soaked with the SAE composition after the air tight seal is broken on the storage compartment.

In some embodiments, the kit for delivering a SAE composition to a subject with an applicator unit, wherein the applicator unit comprises a storage compartment. In some embodiments, the kit for delivering a SAE composition to a subject with an applicator unit, wherein the applicator unit comprises a storage compartment to protect the SAE composition from atmospheric water. In some embodiments, the kit for delivering a SAE composition to a subject, wherein the SAE composition is essentially free of water. In some embodiments, the kit for delivering a SAE composition to a subject, wherein the SAE composition is free of water. In some embodiments, the kit for delivering a SAE composition to a subject, wherein the SAE composition is essentially free of water and DMSO.

In some embodiments, the kit for delivering a SAE composition to a subject, wherein said kit provides for controlled delivery of the SAE composition to the subject. In some embodiments, the kit for delivering a SAE composition to a subject distributes the SAE composition evenly across an area of skin to be treated. In some embodiments, the area of skin to be treated is variable in size. In some embodiments, the maximum area of skin to be treated is about 800 cm². In some embodiments, the controlled delivery of the SAE composition to a subject, wherein said delivery provides a precise volume of the SAE composition. In some embodiments, the controlled delivery of the SAE composition to a subject, wherein said delivery is performed at a predetermined rate. In some embodiments, the controlled delivery of the SAE composition to a subject, wherein said delivery protects the SAE composition from water prior to administration to the subject.

In some embodiments, the kit for delivering a SAE composition to a subject, wherein the SAE composition is administered to a subject in need thereof. In some embodiments, the kit for delivering a SAE composition to a subject, wherein the SAE composition is administered topically to a subject in need thereof. In some embodiments, the kit for delivering a SAE composition to a subject, wherein the SAE composition is administered to the skin of a subject in need thereof. In some embodiments, the system for delivering a SAE composition to a subject, wherein the SAE composition is administered to the scalp of a subject in need thereof. In some embodiments, the kit for delivering a SAE composition to a subject, wherein the SAE composition is administered on a skin lesion of a patient in need thereof to treat a skin condition. In some embodiments, the kit for delivering a SAE composition to a subject, wherein the SAE composition is administered adjacent to a skin lesion of a patient in need thereof to treat a skin condition. In some embodiments, the kit for delivering a SAE composition to a subject, wherein the SAE composition is administered to an area of the skin that is determined to be optimal by a physician. In some embodiments, the kit for delivering a SAE composition to a subject, wherein the SAE composition is administered selectively to an area of skin in need thereof. In some embodiments, the kit for delivering a SAE composition to a subject, wherein the SAE composition does not make contact with any unintended area of skin. In some embodiments, the kit for delivering a SAE composition to a subject, wherein the SAE composition is entirely contained within the storage unit and applicator unit.

In some embodiments, the system for delivering a SAE composition to a subject with a skin disease. In some embodiments, the kit for delivering a SAE composition to a subject with hair loss. In some embodiments, the kit for delivering a SAE composition to a subject with alopecia areata. In some embodiments, the system for delivering a SAE composition to a subject to treat a condition selected from viral warts, herpes simplex virus, molluscum contagiousum, and cutaneous t cell lymphoma.

In some embodiments, the kit for delivering a SAE composition to a subject in a therapeutically effective amount. In some embodiments, the therapeutically effective amount is an amount disclosed herein. In some embodiments, the kit for delivering a SAE composition to a subject leads to a disappearance of the conditions being treated. In some embodiments, the kit for delivering a SAE composition to a subject leads to restoration of hair loss. In some embodiments, the kit for delivering a SAE composition to a subject leads to hair growth on regions of skin in direct contact with the SAE composition. In some embodiments, the kit for delivering a SAE composition to a subject leads to hair growth on regions of skin adjacent to the location of administration of the SAE composition.

In some embodiments, the subject is diagnosed with alopecia areata and successful treatment of this condition results in a one point improvement on any ordinal or interval assessment scale, such as the Physician Global Assessment or the Investigator Global Assessment. In some embodiments, the subject is diagnosed with alopecia areata and successful treatment of this condition results in about 100% restoration of hair growth in the location of the patch is administered. In some embodiments, the subject is diagnosed with alopecia areata and successful treatment of this condition results in at least about 50% increase in hair growth as defined by number of hair follicles with actively growing hair shafts as seen with clinical, trichoscopic, or histopathological assessment in the location of the patch is administered. In some embodiments, successful treatment of alopecia areata with the SAE composition results in an increase in hair growth in the location the composition is administered by a range from about 50% to about 100%, about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 60% to about 100%, about 60% to about 95%, about 60% to about 90%, about 60% to about 85%, about 60% to about 80%, about 60% to about 75%, about 60% to about 70%, about 60% to about 65%, about 70% to about 100%, about 70% to about 95%, about 70% to about 90%, about 70% to about 85%, about 70% to about 80%, about 70% to about 75%, about 80% to about 100%, about 80% to about 95%, about 80% to about 90%, about 80% to about 85%, about 90% to about 100%, about 90% to about 95%, or a value within these ranges. Specific examples may include increasing hair growth as defined by the number of hair follicles with actively growing hair shafts as seen with clinical, trichoscopic, or histopathologic assessment, by about 100%, about 99%, about 95%, about 90%, about 85%, about 80%, about 75%, about 70%, about 65%, about 60%, about 55%, about 50% or a range between any two of these values.

In some embodiments, successful treatment of alopecia areata with the SAE composition results in improvement in the Severity Alopecia Tool (SALT) score in the location the patch is administered by a range from about 50% to about 100%, about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 60% to about 100%, about 60% to about 95%, about 60% to about 90%, about 60% to about 85%, about 60% to about 80%, about 60% to about 75%, about 60% to about 70%, about 60% to about 65%, about 70% to about 100%, about 70% to about 95%, about 70% to about 90%, about 70% to about 85%, about 70% to about 80%, about 70% to about 75%, about 80% to about 100%, about 80% to about 95%, about 80% to about 90%, about 80% to about 85%, about 90% to about 100%, about 90% to about 95%, or a value within these ranges. Specific examples may include improving the Severity Alopecia Tool (SALT) score by about 100%, about 99%, about 95%, about 90%, about 85%, about 80%, about 75%, about 70%, about 65%, about 60%, about 55%, about 50% or a range between any two of these values.

In some embodiments, the kit for delivering a SAE composition to a subject, wherein the SAE composition is delivered to the subject once per month. In some embodiments, the kit for delivering a SAE composition to a subject, wherein the SAE composition is delivered to the subject once per month for four months. In some embodiments, the kit for delivering a SAE composition to a subject, wherein the SAE composition is delivered to the subject once per month for one to 12 months, for one to 11 months, for one to 10 months, for one to nine months, for one to eight months, for one to seven months, for one to six months, for one to five months, for one to four months, for one to three months, for one to two months, a value within these ranges. Specific examples may include one month, two months, three months, four months, five months, six months, seven months, eight months, nine months, 10 months, 11 months, 12 months, or a range between any two of these values.

In some embodiments, the kit for delivering a SAE composition to a subject, wherein the subject is evaluated at the conclusion of four months of treatment for hair regrowth. In some embodiments, the kit for delivering a SAE composition to a subject, wherein the subject is evaluated for hair regrowth at the conclusion of four months of treatment. Subjects that experience hair regrowth at the conclusion of four months of treatment are referred to as “responders.” Subjects that do not experience hair regrowth at the conclusion of four months of treatment are referred to as “non-responders.” Subjects that experience measurable hair regrowth at any amount less than about a 50% SALT score at the conclusion of four months of treatment are referred to as “partial responders.” In some embodiments, the kit for delivering a SAE composition to a subject, wherein the subject is a responder. In some embodiments, the kit for delivering a SAE composition to a subject, wherein the subject is a non-responder. In some embodiments, the kit for delivering a SAE composition to a subject, wherein the subject is a partial responder. In some embodiments, the kit for delivering a SAE composition to a non-responder, wherein the SAE composition is delivered every two weeks. In some embodiments, the kit for delivering a SAE composition to a partial responder, wherein the SAE composition is delivered every two weeks. In some embodiments, the kit for delivering a SAE composition to non-responder or a partial responder, wherein the SAE composition is delivered every week. In some embodiments, the kit for delivering a SAE composition to a non-responder or a partial responder, wherein the SAE composition is delivered twice per week. In some embodiments, the kit for delivering a SAE composition to a non-responder or a partial responder, wherein the SAE composition is delivered every 3 weeks.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Instead, this application is intended to cover any variations, uses, or adaptations of the present teachings and use its general principles. Further, this application is intended to cover such departures from the present disclosure as come within known or customary practice in the art to which these teachings pertain. Many modifications and variations can be made to the particular embodiments described without departing from the spirit and scope of the present disclosure, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

### Further Embodiments

Provided herein is embodiment A, a composition comprising: a squaric acid ester (SAE); an excipient; wherein said composition is essentially free of water and DMSO; and wherein said composition has a volume of 1 milliliter.

In an embodiment B, the composition of embodiment A, wherein the composition is shelf stable for up to about 60 months.

In an embodiment C, the composition of embodiment B, wherein the SAE is squaric acid dibutylester (SADBE) or squaric acid diethylester (SADEE).

In an embodiment D, the composition of embodiment B, wherein the SAE is in an amount of at least about 5% (w/v) SAE.

In an embodiment E, the composition of embodiment B, wherein the SAE is in an amount of at least about 10% (w/v) SAE.

In an embodiment F, the composition of embodiment B, wherein the composition is stored at room temperature.

Provided herein is an embodiment G, a system for delivering a squaric acid ester (SAE) composition to a subject comprising: a storage compartment containing the composition having at least about 2.5% (w/v) SAE and an excipient; and an applicator unit, wherein the applicator unit delivers a volume of about 1 milliliter of the SAE composition.

In an embodiment H, a system of embodiment G, wherein the applicator unit comprises a storage compartment, an air tight seal, a connecting region, and an applicator.

In an embodiment I, a system of embodiment H, wherein the storage compartment protects the SAE composition from atmospheric water.

In an embodiment J, a system of embodiment H, wherein an absorbent membrane is located at the distal end of the storage unit.

In an embodiment K, a system of embodiment J, wherein the absorbent membrane is soaked with the SAE composition after the air tight seal is broken on the storage compartment.

In an embodiment L, a system of embodiment G, wherein said system provides for controlled delivery of the SAE composition to the subject.

In an embodiment M, a system of embodiment G, wherein the SAE composition is administered topically to a subject in need thereof.

Provided herein is an embodiment N, a method of delivering a squaric acid ester (SAE) composition to a subject for treating a condition susceptible to treatment with the SAE composition, comprising: providing applicator device comprising a storage compartment, an air tight seal, a connecting region, and an applicator; and delivering the SAE composition from the storage compartment through the applicator to the subject.

In an embodiment O, a method of embodiment N, wherein the subject is diagnosed with a skin disease.

In an embodiment P, a method of embodiment N, wherein the subject is diagnosed with alopecia areata.

In an embodiment Q, a method of embodiment N, wherein the composition is delivered to the scalp of a subject for the treatment of hair loss.

In an embodiment R, a method of embodiment N, wherein the delivering to a subject results in hair regrowth.

In an embodiment S, a method of embodiment N, wherein the delivering comprises delivering about 1 milliliters of the SAE composition.

In an embodiment T, a method of embodiment N, wherein the SAE composition comprises at least about 5% (w/v).

In an embodiment U, a method of embodiment N, wherein the SAE composition comprises at least about 7% (w/v).

In an embodiment V, a method of embodiment N, wherein the SAE composition comprises at least about 10% (w/v).

In an embodiment W, a method of embodiment N, wherein the delivering of the SAE composition to a subject is a controlled delivery.

In an embodiment X, a method of embodiment N, wherein the delivering of the SAE composition to a subject is a controlled delivery.

In an embodiment Y, a method of embodiment N, wherein the SAE composition is delivered to a subject once per month.

In an embodiment Z, a method of embodiment N, wherein the SAE composition is delivered to the subject once per month for four months.

In an embodiment AA, a method of embodiment Z, wherein the subject is evaluated at the conclusion of the four months for hair regrowth, wherein subjects with hair regrowth are responders.

In an embodiment AB, a method of embodiment Z, wherein the subject is evaluated at the conclusion of the four months for hair regrowth, wherein subjects without hair regrowth are non-responders.

In an embodiment AC, a method of embodiment AB, wherein the nonresponder is delivered the SAE composition every two weeks.

Provided herein is an embodiment AD, a kit for administering a squaric acid ester (SAE) composition to a subject, comprising: an applicator device comprising a storage compartment, an air tight seal, a connecting region, and an applicator; the SAE composition comprising at least 5% SAE; said kit further comprising instructional material for administering said SAE composition for treating a condition susceptible to treatment using said SAE composition.

### EXAMPLES

The following are provided for exemplification purposes only and are not intended to limit the scope of the embodiments described in broad terms above.

### Example 1: SADBE Stability

The stability of SADBE was tested in vehicles with ethyl acetate over a 3-month period of time. The SADBE formulations are characterized in Table 1.

TABLE 1
SADBE Formulations

| | | Vehicle 8 | Vehicle 9 | Vehicle 10 |
|---|---|---|---|---|
| Ingredient | Function | (% w/w) | (% w/w) | (% w/w) |
| SADBE | Active | 5.0 | 5.0 | 5.0 |
| Medium chain | Nonvolatile | 13.0 | 0.0 | 0.0 |
| triglycerides, USP | Solvent | | | |
| Isopropyl | Nonvolatile | 7.0 | 4.0 | 0.0 |
| palmitate, NF | Solvent | | | |
| Diethyl | Nonvolatile | 24.0 | 24.0 | 24.0 |
| sebacate | Solvent | | | |
| Ethyl | Volatile | 15.0 | 31.0 | 50.0 |
| acetate, NF | Solvent | | | |
| Isopropyl | Nonvolatile | q.s. to | q.s. to | q.s. to |
| myristate, NF | Solvent | 100% | 100% | 100% |

The formulations presented in Table 1 were filled into Type 1 glass vials and closed with a butyl rubber stopper and an aluminum crimp seal. The results for initial and up to 3-months of testing are summarized in Table 2 (NT=not tested; * clear, colorless solution with no precipitate or deposition on vial walls).

TABLE 2
SADBE Stability Results

| Test | | Vehicle 8 | Vehicle 9 | Vehicle 10 |
|---|---|---|---|---|
| Initial | | | | |
| | Assay (% w/w) | 5.15 | 5.18 | 5.20 |
| | Impurities | None | None | None |
| | (Area %) | Detected | Detected | Detected |
| | Appearance | Conforms* | Conforms* | Conforms* |
| 1 month | | | | |
| at 40° C. | | | | |
| | Assay (% | 100.6 | 101.2 | 101.5 |
| | initial) | | | |
| | Impurities | None | None | None |
| | (Area %) | Detected | Detected | Detected |
| | Appearance | Conforms* | Conforms* | Conforms* |
| 2 months | | | | |
| at 40° C. | | | | |
| | Assay (% | 101.4 | 102.3 | 103.6 |
| | initial) | | | |
| | Impurities | None | None | None |
| | (Area %) | Detected | Detected | Detected |
| | Appearance | Conforms* | Conforms* | Conforms* |
| 3 months | | | | |
| at 40° C. | | | | |
| | Assay (% | NT | 103.8 | 104.3 |
| | initial) | | | |
| | Impurities | NT | None | None |
| | (Area %) | | Detected | Detected |
| | Appearance | Conforms* | Conforms* | Conforms* |
| 3 months | | | | |
| at 25° C. | | | | |
| | Assay (% | NT | 100.1 | 99.9 |
| | initial) | | | |
| | Impurities | NT | None | None |
| | (Area %) | | Detected | Detected |
| | Appearance | Conforms* | Conforms* | Conforms* |

After 3 months at 40° C., there were significant increases in the assay values for all three formulations. This assay increase was in agreement with the weight loss from the vials. Ethyl acetate is a volatile solvent and the container/closure was not optimized for this ingredient. There were no new impurities detected and the appearance was unchanged. All of the data to date show that SADBE is stable in Vehicles 9 and 10 for up to three months at 40° C.

Preferences and options for a given aspect, feature, embodiment, or parameter of the invention should, unless the context indicates otherwise, be regarded as having been disclosed in combination with any and all preferences and options for all other aspects, features, embodiments, and parameters of the invention.

While present disclosure has been disclosed with reference to various embodiments, it is apparent that other embodiments and variations of these may be devised by others skilled in the art without departing from the true spirit and scope of the disclosure. The appended claims are intended to be construed to include all such embodiments and equivalent variations.

What is claimed is:

1. A composition comprising:
a squaric acid ester (SAE);
an excipient;
wherein said composition is essentially free of water and DMSO; and
wherein said composition has a volume of 1 milliliter.

2. The composition of claim 1, wherein the composition is shelf stable for up to about 60 months.

3. The composition of claim 2, wherein the SAE is squaric acid dibutylester (SADBE) or squaric acid diethylester (SADEE).

4. The composition of claim 2, wherein the SAE is in an amount of at least about 5% (w/v) SAE.

5. The composition of claim 2, wherein the SAE is in an amount of at least about 10% (w/v) SAE.

6. The composition of claim 2, wherein the composition is stored at room temperature.

7. A system for delivering a squaric acid ester (SAE) composition to a subject comprising:
a storage compartment containing the composition having at least about 2.5% (w/v) SAE and an excipient; and
an applicator unit, wherein the applicator unit delivers a volume of about 1 milliliter of the SAE composition.

8. The system of claim 7, wherein the applicator unit comprises a storage compartment, an air tight seal, a connecting region, and an applicator.

9. The applicator unit of claim 8, wherein the storage compartment protects the SAE composition from atmospheric water.

10. The applicator unit of claim 8, wherein an absorbent membrane is located at the distal end of the storage unit.

11. The absorbent membrane of claim 10, wherein the absorbent membrane is soaked with the SAE composition after the air tight seal is broken on the storage compartment.

12. The system of claim 7, wherein said system provides for controlled delivery of the SAE composition to the subject.

13. The system of claim 7, wherein the SAE composition is administered topically to a subject in need thereof.

14. A method of delivering a squaric acid ester (SAE) composition to a subject for treating a condition susceptible to treatment with the SAE composition, comprising:
providing applicator device comprising a storage compartment, an air tight seal, a connecting region, and an applicator; and
delivering the SAE composition from the storage compartment through the applicator to the subject.

15. The method of claim 14, wherein the subject is diagnosed with a skin disease.

16. The method of claim 14, wherein the subject is diagnosed with alopecia areata.

17. The method of claim 14, wherein the composition is delivered to the scalp of a subject for the treatment of hair loss.

18. The method of claim 14, wherein the delivering to a subject results in hair regrowth.

19. The method of claim 14, wherein the delivering comprises delivering about 1 milliliters of the SAE composition.

20. The method of claim 14, wherein the SAE composition comprises at least about 5% (w/v).

21. The method of claim 14, wherein the SAE composition comprises at least about 7% (w/v).

22. The method of claim 14, wherein the SAE composition comprises at least about 10% (w/v).

23. The method of claim 14, wherein the delivering of the SAE composition to a subject is a controlled delivery.

24. The method of claim 14, wherein the SAE composition is delivered to a subject once per month.

25. The method of claim 14, wherein the SAE composition is delivered to the subject once per month for four months.

26. The method of claim 25, wherein the subject is evaluated at the conclusion of the four months for hair regrowth, wherein subjects with hair regrowth are responders.

27. The method of claim 25, wherein the subject is evaluated at the conclusion of the four months for hair regrowth, wherein subjects without hair regrowth are non-responders.

28. The method of claim 27, wherein the nonresponder is delivered the SAE composition every two weeks.

29. A kit for administering a squaric acid ester (SAE) composition to a subject, comprising:
an applicator device comprising a storage compartment, an air tight seal, a connecting region, and an applicator;
the SAE composition comprising at least 5% SAE;
said kit further comprising instructional material for administering said SAE composition for treating a condition susceptible to treatment using said SAE composition.
